(19) United States
(12) Patent Application Publication
(10) Pub. No.: US 2013/0122496 A1
(43) Pub. Date: May 16, 2013

(54) STORAGE OF NUCLEIC ACID

(71) Applicant: Blood Cell Storage, Inc., Seattle, WA (US)

(72) Inventors: Paul V. Haydock, Shoreline, WA (US); Lynn M. Barker, Edmonds, WA (US)

(73) Assignee: BLOOD CELL STORAGE, INC., Seattle, WA (US)

(21) Appl. No.: 13/629,300

(22) Filed: Sep. 27, 2012

Related U.S. Application Data
(60) Provisional application No. 61/542,025, filed on Sep. 30, 2011.

(51) Int. Cl. C12Q 1/68

(52) U.S. Cl. CPC C12Q 1/6806
USPC 435/6.11; 536/23.1; 435/91.2; 435/6.12

(57) ABSTRACT

Processes are disclosed for storing nucleic acid in a stable form. A solution comprising nucleic acid is applied to an unmodified, silica-based substrate whereby at least a portion of the nucleic acid binds to a surface of the substrate, the bound nucleic acid is washed and dried, and the resulting dried nucleic acid on the substrate is stored at from 5° C. to 60° C. for a period of at least one week.

### CROSS-REFERENCE TO RELATED APPLICATION

This application claims the benefit of U.S. Provisional Application No. 61/542,025, filed Sep. 30, 2011, which disclosure is incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 40144_SEQ_Final.txt. The text file is 1.82 KB; was created on Sep. 27, 2012; and is being submitted via EFS-Web with the filing of the specification.

### BACKGROUND OF THE INVENTION

In the field of molecular biology, including such applications as nucleic acid analysis and genetic testing, it is often necessary or desirable to store nucleic acid samples for a period of time between collection and analysis or testing. It is also sometimes desirable to store backup samples for later confirmatory analysis or follow-up. It may also be necessary to ship samples from the point of collection to the testing facility.

Following collection, biological samples (including nucleic acids) must be protected from degradation and contamination. Biological samples are often stored and shipped refrigerated or frozen (e.g., nucleic acids are commonly stored at −20° C.), but such conditions can be difficult to ensure when operating in remote locations. To permit storage and shipping of nucleic acid samples at ambient temperature, several stabilizing compositions have been disclosed. See, for example, Whitney et al., U.S. Patent Application Publication No. 20110081363 A1; Hogan et al., U.S. Patent Application Publication No. 20100248363 A1; and Burgoyne, U.S. Pat. No. 5,756,126.

There remains a need in the art for simplified, low-cost methods of storing nucleic acids while protecting against degradation and contamination. The present invention provides such methods, as well as other, related advantages.

### SUMMARY OF THE INVENTION

Within one aspect, the present invention provides a process for storing nucleic acid in a stable form. The processes comprise applying a solution comprising nucleic acid to an unmodified, silica-based substrate whereby at least a portion of the nucleic acid binds to a surface of the substrate to produce bound nucleic acid; washing the bound nucleic acid with a wash solution to produce substrate-bound, washed nucleic acid; drying the substrate-bound, washed nucleic acid to produce dried nucleic acid; and storing the dried nucleic acid on the substrate at a temperature of from 5° C. to 60° C. for a period of at least one week. Within one embodiment, the nucleic acid is DNA. Within another embodiment, the nucleic acid is RNA. Within an additional embodiment, the solution further comprises a chaotropic salt. Within another embodiment, the wash solution comprises an aliphatic C1 to C4 alcohol. Within related embodiments, the wash solution comprises ethanol or methanol. Within other embodiments, the washing step comprises washing the bound nucleic acid with a first wash solution and a final wash solution, wherein the final wash solution does not comprise a mineral salt. Within additional embodiments, the silica substrate is glass. is smooth glass. Within related embodiments, the glass is smooth glass. Within other related embodiments, the smooth glass is in sheet form or in tubular form. Within another embodiment, the smooth glass is a portion of an inner surface of a device, the device comprising the inner surface, an outer surface, a first port, and a second port, wherein the inner surface defines a binding chamber providing fluid communication between the first port and the second port. Within another embodiment, the substrate is in bead form. Within a further embodiment, the substrate is a silica gel membrane. Within an additional embodiment, following the storing step, the nucleic acid is eluted from the glass substrate. Within a related embodiment, the eluted nucleic acid is amplified. Within another embodiment, the nucleic acid is stored for at least two weeks. Within other embodiments, the nucleic acid is stored for up to six months, up to one year, up to two years, or up to five years. Within a further embodiment, the nucleic acid is stored at a temperature of from 15° C. to 30° C.

Within a second aspect of the invention there is provided a process for storing nucleic acid in a stable form, consisting of applying a solution comprising nucleic acid to an unmodified, silica-based substrate whereby at least a portion of the nucleic acid binds to a surface of the substrate to produce bound nucleic acid; washing the bound nucleic acid with a wash solution to produce substrate-bound, washed nucleic acid; drying the substrate-bound, washed nucleic acid to produce dried nucleic acid; and storing the dried nucleic acid on the substrate at a temperature of from 5° C. to 60° C. for a period of at least one week. Within one embodiment, the nucleic acid is stored for at least two weeks.

These and other aspects of the invention will become evident upon reference to the following detailed description of the invention and the attached drawings.

All references cited herein are incorporated by reference in their entirety. Numeric ranges recited herein include the endpoints.

### BRIEF DESCRIPTION OF THE DRAWINGS

FIG. 1 illustrates a representative nucleic acid capture and storage device that can be used within the present invention.

FIG. 2 illustrates the results of an experiment wherein DNA was bound to a smooth glass substrate and stored at room temperature for up to six months.

FIG. 3 illustrates the results of an accelerated stability study of DNA bound to a smooth glass substrate. E1, E2, and E3 are first, second, and third eluates, respectively.

FIG. 4 illustrates amplification results of DNA samples from an accelerated stability study.

FIG. 5 illustrates the results of an experiment wherein RNA was bound to a smooth glass substrate and stored at room temperature for up to fourteen days.

FIG. 6 illustrates the results of an experiment wherein RNA was bound to a smooth glass substrate and stored at room temperature for up to four weeks.

FIG. 7 compares dry storage of Bacteriophase MS2 RNA at room temperature as a function of time.

### DESCRIPTION OF THE INVENTION

The present invention is based in part on the discovery that nucleic acid, following extraction from a biological sample by binding to an unmodified, silica-based substrate, can be dried and stored for extended periods without the need for refrigeration or freezing. The nucleic acid is stored without coating or encapsulation, and without the addition of stabilizing compounds or solutions. The present invention thus provides for the long-term storage of dried nucleic acid over a broad range of ambient temperatures. DNA has been found to be particularly stable when stored according to the present invention.

“Nucleic acid” includes deoxyribonucleic acid (DNA), ribonucleic acid (RNA), and mixtures thereof, including naturally occurring and synthesized forms. “Nucleic acid” includes chromosomal and extrachromosomal forms, and fragments thereof. The singular form of the term includes mixtures of molecules, including molecules of varying size, source, and chemical composition (including mixtures of DNA and RNA).

The term “stable,” when used to describe stored nucleic acid, means that the nucleic acid can be amplified, typically by quantitative polymerase chain reaction (PCR). Quantitative PCR results are stated in terms of Ct values using a logarithmic scale wherein a change of 3 Ct units indicates a 10-fold change in quantity. “Stable” nucleic acid is at least 10% intact by this test. Higher levels of stability are preferred, particularly when the nucleic acid is at a low concentration in the source sample and the yield is low. Within certain embodiments of the invention, the nucleic acid is at least 50% intact, at least 60% intact, at least 70% intact, or at least 80% intact; and may be 90% or more intact.

As used herein, the term “biological sample” means a sample containing cells or cell components and includes any sample, liquid or solid, that contains nucleic acid. Suitable biological samples that can be used within the invention include, without limitation, cell cultures, culture broths, cell suspensions, tissue samples, cell lysates, cleared cell lysates, whole blood, serum, plasma, platelet-rich plasma or other blood fractions, buffy coat, urine, feces, cerebrospinal fluid, semen, saliva, wound exudate, tissue biopsies, mucus, viruses, mitochondria, chloroplasts, water samples, soil samples, and extracts of meat or other foodstuffs.

The term “capture device” is used herein to denote a device used to isolate nucleic acid by binding it to a solid substrate. The present invention makes use of unmodified, silica-based substrates. The substrate will commonly be substantially enclosed within the capture device, although other arrangements are possible.

As used herein, an “unmodified, silica-based substrate” is a silica-based substrate wherein the surface (or surfaces) thereof has (have) not been chemically modified to enhance nucleic acid binding or stability. In particular, an unmodified, silica-based substrate is essentially free of nucleic acid-specific binding sites, that is, it does not contain an amount of such sites sufficient to give a statistically significant increase in nucleic acid binding as compared to the substrate in native form. Examples of nucleic acid-specific binding sites commonly used in the art and specifically excluded from the present invention include charged surfaces and binding sites provided by immobilized oligonucleotides, minor groove binding agents, intercalating agents, and the like. Unmodified, silica-based substrates are also free of coatings designed to stabilize nucleic acids, such as coatings containing urate, urate salts, or other free radical traps as disclosed by Burgoyne, U.S. Pat. No. 5,807,527.

Silica-based substrates suitable for use within the present invention include silica (SiO₂), glass, quartz, diatomaceous earth, silicates (including, for example, aluminum silicate and silica gel), amorphous silicon oxide, alkyl silica, and other such substrates known in the art to bind nucleic acids. See, for example, U.S. Pat. Nos. 6,821,757; 7,238,530; and 8,026,068. Substrates may be provided in a variety of forms, including sheet, tube, bead, powder, frit, membrane, fiber, and gel form as known in the art.

“Smooth” glass means glass having a surface smoothness corresponding to that of a standard microscope slide, Pasteur pipette, glass capillary, or the like, wherein the surface has not been etched or otherwise altered to increase its surface area. Specifically excluded from “smooth glass” is porous glass that is known in the art to capture nucleic acid, commonly in bead, frit, or membrane form. Such porous glass commonly has pores sized within the range of 0.1 μm to 300 μm. Examples of smooth glass materials for use within the present invention include soda lime glass (e.g., Erie Electroverre Glass; Erie Scientific Company, Portsmouth, N.H.), borosilicate glass (e.g., Corning 0211, PYREX 7740; Corning Incorporated, Corning, N.Y.), zinc titania glass (Corning), and silica glass (e.g., VYCOR 7913; Corning Incorporated). Smooth glass is readily utilized in sheet or tubular form. Suitable for use within the invention is glass tubing, which is readily available in a variety of sizes. Of particular interest are Pasteur pipettes, which are inexpensive, provide a good surface:volume ratio, and include a large diameter region within the lumen to facilitate mixing of reagents. Glass capillaries, chromatography columns, condenser tubes, syringes, rods, and the like having smooth glass surfaces can also be employed. Suitable sheet materials include glass microscope slides, which are available in multiple sizes (commonly approximately 1″×3″ and 2″×3″) and glass coverslips, also available in multiple sizes, although larger sheet materials can also be utilized.

The present invention provides processes for storage of nucleic acid for periods of one week or more. The processes of the invention can be used to store nucleic acid for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks. Therefore, using the processes of the present invention, nucleic acid can be stored for up to 4 weeks, up to 8 weeks, up to 12 weeks, up to 16 weeks, up to 20 weeks, up to 24 weeks, up to 28 weeks, up to 32 weeks, up to 36 weeks, up to 40 weeks, up to 44 weeks, up to 48 weeks, or up to 52 weeks. The nucleic acid can be stored for longer than a year, such as for 18 months, 2 years, 2.5 years, 3 years, 3.5 years, 4 years, 5 years, 10 years, 20 years, or up to 100 years.

Within certain embodiments of the invention, nucleic acid is stored for from 1 week to 2 years, from 1 week to 18 months, from 1 week to 1 year, from 1 to 48 weeks, from 1 to 44 weeks, from 1 to 40 weeks, from 1 to 36 weeks, from 1 to 32 weeks, from 1 to 28 weeks, from 1 to 24 weeks, from 1 to 20 weeks, from 1 to 16 weeks, from 1 to 12 weeks, from 1 to 8 weeks, or from 1 to 4 weeks. Within certain other embodiments of the invention, nucleic acid is stored for from 2 weeks to 2 years, from 2 weeks to 18 months, from 2 weeks to 1 year, from 2 to 48 weeks, from 2 to 44 weeks, from 2 to 40 weeks, from 2 to 36 weeks, from 2 to 32 weeks, from 2 to 28 weeks, from 2 to 24 weeks, from 2 to 20 weeks, from 2 to 16 weeks, from 2 to 12 weeks, from 2 to 8 weeks, or from 2 to 4 weeks. Within other embodiments of the invention, nucleic acid is stored for from 3 weeks to 2 years, from 3 weeks to 18 months, from 3 weeks to 1 year, from 3 to 48 weeks, from 3 to 44 weeks, from 3 to 40 weeks, from 3 to 36 weeks, from 3 to 32 weeks, from 3 to 28 weeks, from 3 to 24 weeks, from 3 to 20 weeks, from 3 to 16 weeks, from 3 to 12 weeks, from 3 to 8 weeks, or from 3 to 4 weeks. Within additional embodiments of the invention, nucleic acid is stored for from 4 weeks to 2 years, from 4 weeks to 18 months, from 4 weeks to 1 year, from 4 to 48 weeks, from 4 to 44 weeks, from 4 to 40 weeks, from 4 to 36 weeks, from 4 to 32 weeks, from 4 to 28 weeks, from 4 to 24 weeks, from 4 to 20 weeks, from 4 to 16 weeks, from 4 to 12 weeks, from 4 to 8 weeks, or from 4 to 6 weeks. Within other embodiments of the invention, nucleic acid is stored for from 6 weeks to 2 years, from 6 weeks to 18 months, from 6 weeks to 1 year, from 6 to 48 weeks, from 6 to 44 weeks, from 6 to 40 weeks, from 6 to 36 weeks, from 6 to 32 weeks, from 6 to 28 weeks, from 6 to 24 weeks, from 6 to 20 weeks, from 6 to 16 weeks, from 6 to 12 weeks, or from 6 to 8 weeks. Within additional embodiments of the invention, nucleic acid is stored for from 8 weeks to 2 years, from 8 weeks to 18 months, from 8 weeks to 1 year, from 8 to 48 weeks, from 8 to 44 weeks, from 8 to 40 weeks, from 8 to 36 weeks, from 8 to 32 weeks, from 8 to 28 weeks, from 8 to 24 weeks, from 8 to 20 weeks, from 8 to 16 weeks, or from 8 to 12 weeks.

The present invention provides for storage of dried nucleic acid above refrigerator temperature (i.e., greater than 4° C.) for extended periods of time. Nucleic acid will typically be stored at room temperature (15° C.-30° C., often 18-25° C., and more commonly 20-22° C.), although storage at higher or lower temperatures, such as those likely to be encountered in conducting fieldwork, in developing countries, under battlefield conditions, or during shipment, is within scope of the present invention. Storage temperatures up to 37° C., up to 45° C., and up to 60° C. are within the scope of the present invention, although storage times above room temperature should be limited to the extent practicable. The invention thus provides for storage at temperatures of 5° C. to 60° C., 5° C. to 45° C., 5° C. to 37° C., 5° C. to 30° C., 5° C. to 25° C., 5° C. to 22° C., 5° C. to 20° C., 5° C. to 15° C., or 5° C. to 10° C. In certain embodiments of the invention, nucleic acid is stored at temperatures of 10° C. to 60° C., 10° C. to 45° C., 10° C. to 37° C., 10° C. to 30° C., 10° C. to 25° C., 10° C. to 22° C., 10° C. to 20° C., or 10° C. to 15° C. Within other embodiments, nucleic acid is stored at temperatures of 15° C. to 60° C., 15° C. to 45° C., 15° C. to 37° C., 15° C. to 30° C., 15° C. to 25° C., 15° C. to 22° C., or 15° C. to 20° C. Within further embodiments, nucleic acid is stored at temperatures of 20° C. to 60° C., 20° C. to 45° C., 20° C. to 37° C., 20° C. to 30° C., or 20° C. to 25° C. Within additional embodiments, nucleic acid is stored at temperatures of 25° C. to 60° C., 25° C. to 45° C., 25° C. to 37° C., 25° C. to 30° C., 30° C. to 60° C., 30° C. to 45° C., 30° C. to 37° C., 37° C. to 45° C., 37° C. to 60° C., or 45° C. to 60° C.

Within the above-disclosed ranges of storage parameters, the stored nucleic acid can be DNA, RNA, or a mixture thereof.

The dried nucleic acid is essentially free of stabilizers. As used herein, the term “stabilizers” denotes agents known in the art to reduce the degradation of stored nucleic acid. Examples of such agents include borate compounds, zwitterionic compounds, and osmoprotectants as disclosed by Whitney et al., U.S. Application Publication No. 20110081363; and singlet oxygen quenchers as disclosed by Hogan et al., U.S. Application Publication No. 20100248363. The term “stabilizers” is not intended to include agents that specifically retard or inhibit the growth of microorganisms. Antimicrobial agents in this regard include sodium azide and isothiazolinone preservatives. An exemplary such antimicrobial agent is a mixture of the isothiazolinones 2-Methyl-4-isothiazolin-3-one, 5-Chloro-2-methyl-4-isothiazolin-3-one, and 1,2-Benzisothiazolin-3-one, which is commercially available from Sigma-Aldrich, Inc., St. Louis, Mo. under the name PROCLIN. Other isothiazolinone preservatives are known in the art (e.g., KATHON; Rohm and Haas Co.).

Methods for capturing nucleic acid on glass and other silica-based substrates are known in the art. See, for example, Boom et al., U.S. Pat. No. 5,234,809; Reed et al., U.S. Pat. No. 7,608,399; Reed et al., U.S. Application Publication No. 20110203688 A1; Reed et al., U.S. Pat. No. 8,163,535; and Kim et al., Proc. of the IEEE 15th Annual Int. Conf. on MEMS 2002, 133-136, 2002. Nucleic acid in solution is captured on a variety of silica-based surfaces, including smooth glass sheets or tubing, porous glass beads, etched glass surfaces, filters, fibers, silica-gel membranes, and the like. Extraction devices and reagents are available from commercial suppliers (e.g., QIAGEN, Valencia, Calif.). In a widely used procedure, a nucleic acid-containing sample in a chaotropic salt solution is applied to a glass substrate to allow the nucleic acid to bind. The substrate is then washed using buffered solutions of decreasing salt concentration, usually containing increasing amounts of ethanol as the salt concentration decreases. Following a final, ethanol-rich wash, the substrate can be dried, such as by flowing a stream of air over or through the substrate, or by using a vacuum pump. Variations on this basic protocol are known in the art; exemplary methods are disclosed in more detail below.

As noted above, biological samples containing cells or cellular components are suitable as a source of nucleic acid for use within the invention. Dilute or concentrated samples can be applied to the silica-based substrate. Nucleic acid can be extracted from a wide variety of sources. For research and medical applications, suitable sources include, without limitation, sputum, saliva, throat swabs, oral rinses, nasopharyngeal swabs, nasopharyngeal aspirates, nasal swabs, nasal washes, mucus, bronchial aspirations, bronchoalveolar lavage fluid, pleural fluid, endotracheal aspirates, cerbrospinal fluid, feces, urine, blood, plasma, serum, cord blood, blood components (e.g., platelet concentrates), blood cultures, peripheral blood mononuclear cells, peripheral blood leukocytes, plasma lysates, leukocyte lysates, buffy coat leukocytes, anal swabs, rectal swabs, vaginal swabs, endocervical swabs, semen, biopsy samples, lymphoid tissue (e.g., tonsil, lymph node), bone marrow, other tissue samples, bacterial isolates, vitreous fluid, amniotic fluid, breast milk, and cell culture supernatants. Other starting materials for extraction of nucleic acid include water samples, air samples, soil samples, cosmetics, foods and food ingredients, medical supplies and equipment, and the like. Lysis and digestion of intact cells releases DNA and/or RNA from residual proteins (for example histones). In the alternative, solid samples (e.g., bacterial spores or dried blood on cloth) or semisolid samples (e.g., mouse tails or sputum/stool) can be homogenized and lysed before input to the capture device to provide a homogeneous and non-viscous sample that will flow through the device and over the silica-based substrate. More viscous samples, such as blood, can also be used.

In general, when cells are present within the biological sample they are lysed to provide a cell lysate from which the nucleic acid is extracted. A variety of methods of cell lysis are known in the art and are suitable for use within the invention. Examples of cell lysis methods include enzymatic treatment (using, for example, proteinase K, pronase, or subtilisin), mechanical disruption (e.g., by sonication, application of high pressure, use of a piezobuzzer device, or bead beating), or chemical treatment. Beads used for mechanical disruption should be made of a substance that does not bind nucleic acid under the disruption conditions. Suitable substances include acrylic, polycarbonate, polypropylene, cellulose acetate, polyethylene terephthalate, polyvinylchloride, and high density polyethylene. Lysing cells in the sample by treating them with a chaotropic salt solution is particularly advantageous. Methods and reagents for lysing cells using chaotropic salts are known in the art, and reagents can be purchased from commercial suppliers. Lysis is generally carried out between room temperature and about 95° C., depending on the cell type. Blood cells are conveniently lysed at room temperature. It is generally preferred that the use of silica particles in cell lysis be avoided, since silica particles may bind nucleic acid and reduce the efficiency of the extraction process. Although not necessary, DNA may be sheared prior to loading the lysate into the extraction device. Methods for shearing DNA are known in the art. Specific reagent compositions and reaction conditions will be determined in part by the type of cell to be lysed, and such determination is within the level of ordinary skill in the art.

Nucleic acid is bound to the surface(s) of the silica-based substrate in the presence of a high concentration of salt. Chaotropic salts are commonly used, particularly when cell lysis is necessary prior to extraction of the nucleic acid. Lysis in a chaotropic salt solution also removes histone proteins from genomic DNA and inactivates nucleases. Commonly used chaotropic salts include guanidine HCl or guanidine thiocyanate, which are used at a concentration of at least 1 M to about 6 M or up to the limit of solubility. Guanidine thiocyanate is preferred over guanidine hydrochloride when the nucleic acid will later be amplified by polymerase chain reaction. In the alternative, other salts (e.g., KCl) at a concentration of at least 0.5 M to about 2 M or more depending on solubility, may be used in place of the chaotrope. A C1 to C4 aliphatic alcohol, such as methanol, ethanol, isopropanol, or tert-butyl alcohol, can be included in the binding solution. Binding of nucleic acid is ordinarily done at a pH of approximately 5 to 8, preferably about 6. Suitable buffers include Tris(tris(hydroxymethyl)aminomethane), citrate, phophate, and MES (2-(N-morpholino)ethanesulfonic acid). The substrate-bound nucleic acid is then washed using buffered solutions at pH=5-9 of decreasing salt concentration. As salt concentration decreases, ethanol is commonly added to the wash solution to retain the nucleic acid on the substrate and to remove contaminants that may interfere with downstream processes such as nucleic acid amplification. Washing is carried out at pH 6-9, commonly pH 6-8. Nucleic acid are eluted from the device with a low-salt solution at basic pH, commonly pH 8-9.

Nucleic acid is captured on the silica-based substrate surface(s) in the presence of a salt or chaotropic salt as disclosed above. Satisfactory binding of nucleic acid to the substrate is achieved at room temperature (15° C.-30° C., commonly about 20° C.), although the extraction process can be run at higher temperatures, such as up to 37-42° C. or up to 56° C., although higher temperatures may reduce recovery of nucleic acid. The sample may be allowed to stand in the device for a period of time, and the sample solution may be pumped back and forth over the substrate. Contact times of fifteen to thirty minutes are generally sufficient for nucleic acid binding, although shorter or longer periods (e.g., five minutes up to several hours) may be used. Large samples may be applied to the substrate in a series of aliquots, or, if the capture device permits, flowed slowly over the substrate to achieve the desired contact time.

Suitable chaotropic salts include guanidinium thiocyanate, guanidine hydrochloride, sodium iodide, and sodium perchlorate. Guanidine hydrochloride, which is preferred for lysing blood cells, is used at concentrations of 1M to 10M, commonly 1M to 5M, usually 1M to 3M. Higher concentrations of sodium iodide are required, approaching the saturation point of the salt (12M). Sodium perchlorate can be used at intermediate concentrations, commonly around 5M. As noted above, neutral salts such as potassium chloride and sodium acetate can also be used to obtain binding of nucleic acid to silica-based substrates, and may be used in place of chaotropic salts when cell lysis is not required or is achieved by other means (e.g., in the case of bacterial cell lysis). When using neutral salts, the ionic strength of the buffer should be at least 0.25M. Lysis/binding buffers will generally also contain one or more buffering agents to maintain a near-neutral to slightly acidic pH. A suitable buffering agent is sodium citrate. One or more detergents may also be included. Suitable detergents include, for example, polyoxyethylenesorbitan monolaurate (TWEEN 20), t-octylphenoxypolyethoxyethanol (TRITON X-100), sodium dodecyl sulfate (SDS), NP-40, Cetyltrimethylammonium bromide (CTAB), 3-[(3-Cholamidopropyl)dimethylammonio]-1-propanesulfonate (CHAPS), and sarkosyl. Alcohol, commonly ethanol, may also be included in the lysis buffer. One exemplary lysis/binding buffer is a solution of 4M Guanidine thiocyanate, 50 mM Tris-Cl pH 6.0, 20 mM EDTA, 1% Triton X-100, and 10% Tween-20, which is combined at a ratio of two parts buffer to one part sample to yield final concentrations of 2.7M Guanidine thiocyanate, 33 mM Tris-Cl pH 6.0, 13 mM EDTA, 0.67% Triton X-100, and 6.7% Tween-20. This buffer is preferred for use with plasma samples and bacteria. A second exemplary lysis/binding buffer is a solution of 6M Guanidine hydrochloride, 50 mM sodium citrate pH 6.0, 20 mM EDTA, 3% Triton X-100, and 10% Tween-20, which is combined at a ratio of one part buffer, one part sample, and one part ethanol to yield final concentrations of 2M Guanidine hydrochloride, 16.5 mM sodium citrate pH 6.0, 6.6 mM EDTA, 1% Triton X-100, 3.3% Tween-20, and 33% ethanol. This second buffer is preferred for use with blood products and viral samples.

The bound nucleic acid is then washed to remove contaminants while keeping the nucleic acid bound to the substrate. Wash buffers and protocols are designed to retain most or all of the nucleic acid bound to the substrate while removing detergents, protein and other unwanted cell components, and other contaminants, including any residual chaotropic salt, that can interfere with downstream applications (e.g., nucleic acid amplification). Wash buffers themselves must also be readily removable from the substrate-bound nucleic acid. Selection of wash buffers will depend in part on the composition of the sample loading solution. In general, salt concentration will be reduced during the washing process, and pH will be increased slightly. The pH range for washes is pH 5 to pH 10, with initial washes commonly at a pH of about 6.0 and final washes commonly at a pH of about 7.0. If the lysis buffer contains a chaotropic salt, the initial wash will commonly also contain that salt at the same or somewhat lower concentration (e.g., 1-3M GuSCN). In a typical procedure, the bound nucleic acid is washed with a sequence of two or more buffers of progressively lower ionic strength and increasing concentration of a C1 to C4 aliphatic alcohol (e.g., methanol, ethanol, isopropanol, or tert-butyl alcohol), with the actual alcohol concentration selected to compensate for the lowered salt concentration in the washes. In the absence of salt, alcohol is included at a concentration of at least 50%, with 70% alcohol preferred in the final wash. If salt is included in the reagents, alcohol concentration will ordinarily range between 0% and 80%, often between 10% and 60%, usually between 20% and 50%. In the alternative, higher salt washes without alcohol may be used, however the salt will form a residue on the substrate and result in a high salt concentration in the eluted nucleic acid. Such high salt concentration may interfere with later analysis of the nucleic acid, such as by PCR. Wash buffers containing substantial levels of alcohol also facilitate drying of the substrate and bound nucleic acid while helping to remove residual chaotropic salt and retain the bound nucleic acid on the substrate. Shorter-chain alcohols (e.g., methanol and ethanol) are advantageous insofar as their lower vapor pressures allow for reduce drying times. Optimization of buffers is within the level of ordinary skill in the art. A typical washing procedure comprises two washes with a first buffer similar in composition to the lysis/binding buffer; followed by a series of washes with a second, low-salt, high-alcohol buffer Exemplary wash buffers include 2M Guanidine hydrochloride, 33 mM sodium citrate pH 6.0, 6.7 mM EDTA, 33% ethanol (first wash) and 30 mM Tris-Cl pH 6.0, 0.03% PROCLIN (isothiazolone-based preservative; Sigma-Aldrich, St. Louis, Mo.), 70% ethanol (second wash). These exemplary buffers use either high salt (first wash) or alcohol (second wash) to retain the bound nucleic acid.

Following drying, it is preferred to seal the storage device to protect the nucleic acid from contamination. Adhesive seals or plugs are conveniently used. Ports of DNA cards as disclosed herein can be sealed with adhesive tape. The substrate with the bound, washed, and dried nucleic acid is stored for the desired period of time. Room temperature storage and low humidity are generally preferred.

After a period of storage, the nucleic acid may be eluted from the capture device with a low salt buffer at higher pH than the final wash. Elution buffers are typically low ionic strength, buffered solutions at pH >8.0, although nucleic acid can be eluted from the device with water. Elution can be carried out at ambient temperature up to about 56° C. An exemplary elution buffer is 10 mM Tris-Cl pH 8.0, 1 mM EDTA, 0.05% sodium azide (preservative). In the alternative, the nucleic acid may be eluted from the substrate but retained within the capture device for subsequent analysis or processing. Multi-functional, lab-on-a-chip devices are known in the art.

Nucleic acid captured on silica-based substrates often co-purifies with significant amounts of protein, particularly when starting with protein-rich samples such as blood or blood fractions. For example, DNA extraction from blood using a DNA card as disclosed herein may result in a 10,000- to 20,000-fold purification of DNA over protein, yet the protein content of the bound material may still exceed that of DNA by two or more orders of magnitude by mass. However, the binding and washing procedures disclosed above maintain residual bound proteins in a denatured state so as to minimize degradation of the bound nucleic acid.

Within the present invention, devices comprising smooth glass binding surfaces are preferred for capture and storage of nucleic acid. Smooth-glass devices provide several advantages over those comprising porous capture surfaces. Smooth glass devices are more resistant to clogging, are easier to wash, release the bound nucleic acid more readily, and are generally simpler and cheaper to construct. Such devices typically comprise a plurality of ports and a binding chamber intermediate and in fluid communication with at least two of the plurality of ports. These ports provide for the introduction of a nucleic acid-containing sample into the binding chamber, for the introduction of reagents, and for the removal of waste products and eluted nucleic acid. The binding chamber comprises an unmodified smooth glass surface effective for binding a heterogeneous population of nucleic acid. The glass surface may be flat or curved, the latter including tubular devices where the nucleic acid is captured on an inner wall of the tube. Devices of this type are disclosed in Reed et al., U.S. Pat. No. 7,608,399; Reed et al., U.S. Pat. No. 8,163,535; Reed et al., U.S. Application Publication No. 20080038740 A1; and Reed et al., U.S. Application Publication No. 20110203688 A1. These devices include simple, microtiter plate-sized nucleic acid extraction devices that comprise glass microscope slides as the glass substrate. This type of device (“DNA card”) works especially well with dilute samples that have large volume and low nucleic acid content, including respiratory secretions. The devices disclosed by Reed et al. can be manually operated using pipettors, can be adapted to a gravity-driven system, or can be automated with pumps. The DNA cards comprise (i) a body member having a plurality of external surfaces and fabricated to contain a continuous fluid pathway therethrough, the pathway comprising a first port, a second port, and a binding channel intermediate and in fluid communication with the first port and the second port, wherein the binding channel is open to one of the external surfaces of the body member; and (ii) a glass member affixed to the one of the external surfaces of the body member to provide a first unmodified flat glass surface in fluid communication with the binding channel. The binding channel may be open to a second of the external surfaces of the body member, in which case the device further comprises a second glass member affixed to the second external surface of the body member to provide a second unmodified flat glass surface in fluid communication with the binding channel. Nucleic acid is captured on the glass surface(s) Such devices are conveniently fabricated by lamination of alternating polymeric and adhesive layers according to known methods, and glass microscope slides or cover slips are used as the glass members. To reduce the number of layers the binding channel and ports can be molded into a central member that is joined to one or more glass outer walls using adhesive or compression. See, for example, Reed et al., U.S. Pat. No. 8,163,535. The design of such devices permits fluids, including both liquids and gasses, to be passed through the device from one port to another. In this way buffers can be pumped back and forth through the binding channel to increase washing and elution efficiency, and air can be pumped through between washes and after the final wash to remove residual buffer and to dry the bound nucleic acid. The device can be configured in a variety of ways with respect to introduction and removal of reagents, such as by varying the number and position of access ports.

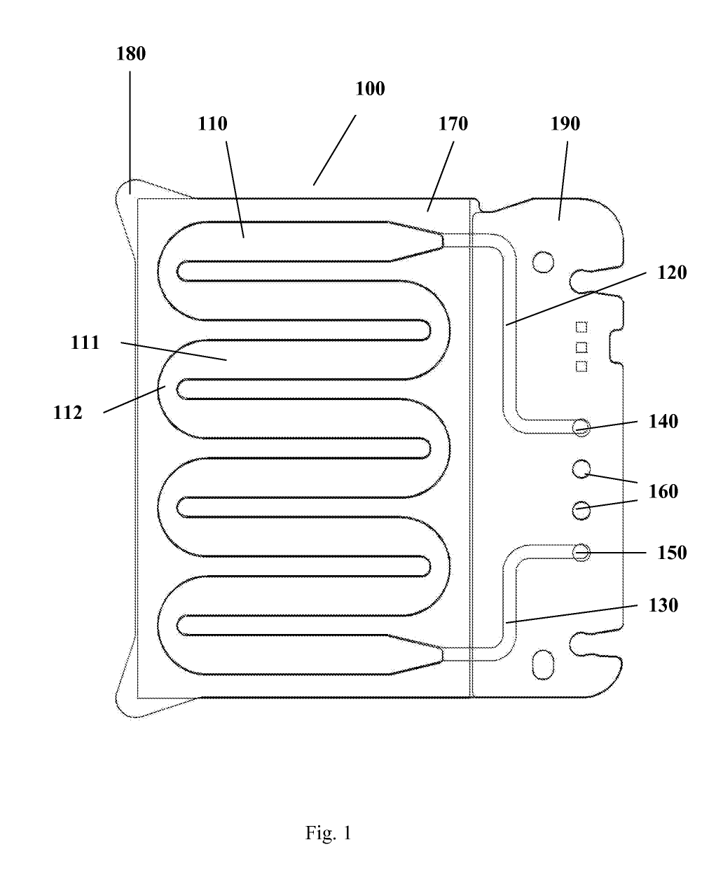

An example of a DNA card for use within the present invention is illustrated in FIG. 1. Device 100 comprises an unmodified, flat glass substrate 170 for nucleic acid binding and is also adapted for use with an optional manifold that can connect to a plurality of such devices. In the illustrated embodiment, flat glass substrate 170 is a 2×3 inch microscope slide, which, together with surface element 190, provides the illustrated external surface. Device 100 comprises S-shaped binding chamber 110, in which linear segments 111 are wider than bends 112. This device further comprises first and second channels 120 and 130, respectively, through which fluids are introduced into and removed from the device. First channel 120 is accessed via first port 140. Second channel 130 is accessed via second port 150. One or both of first port 140 and second port 150 may be equipped with a pipette interface (not shown) to receive and seal to a disposable pipette tip. A plurality of additional channels 160 pass through the device, which channels may be used to join the device to additional components. The illustrated device can be provided with an identification tag (not shown), such as a barcode or RF tag, for sample tracking. The illustrated arrangement allows flow-through operation of the device, wherein liquids are introduced via one of the ports and withdrawn via the other of the ports. In the alternative, the device may be operated in a back-and-forth mode wherein reagents are introduced and withdrawn via one of the ports, and the other of the ports is used exclusively for withdrawal of the final sample so as to reduce the chance for contamination of eluted nucleic acid.

Device 100 may be constructed by laminating a plurality of individual, planar elements to form body member 180, to which glass substrate 170 is attached. Individual elements are joined using silicone adhesive. Material thicknesses and the number of layers can be varied to obtain different device thicknesses and volumes. In one embodiment, a glass substrate 170 is used on each of the front and back faces of the device. In this embodiment, both external layers of device 100 are formed by the combination of the glass substrates 170 with elements 190. Other devices of this type are disclosed by Reed et al., U.S. Pat. No. 8,163,535.

Nucleic acid can also be captured and stored on porous or rough (etched) glass surfaces. Devices and methods for capturing DNA on porous or rough glass are known in the art. For example, Kim et al. (ibid.) disclose a microfluidic device comprising a nucleic acid binding chip constructed from photosensitive glass that is chemically etched to produce a plurality of microchannels separated by pillars. Boom et al. (ibid.) disclose methods for purifying nucleic acid using suspensions of silica particles. Braman et al., U.S. Application Publication No. 20080153078 A1 disclose a lab-on-chip device wherein nucleic acid is captured on a glass fiber filter. A variety of other devices employing magnetic particles, filters, beads, and nanoengineered surfaces for nucleic acid capture are known in the art (reviewed by Malic et al., Recent Patents in Engineering 1:71-88, 2007). Spin columns comprising a silica gel membrane for nucleic acid capture are commercially available (QIAGEN, Valencia, Calif.)

The use of devices that permit flow-through operation is generally preferred for nucleic acid capture due to ease of operation, including reduction of sample handling and adaptability to automation. A variety of such microfluidic devices are known in the art as disclosed above. In general, reagents are pumped into the device through an inlet port, such as by use of a peristaltic pump, a syringe, a pipetter, or a vacuum pump. Devices with integrated pumps are also known (e.g., Quake et al., U.S. Application Publication No. 20020025529 A1). Methods for removal of the final wash include drying by passaging air over the surfaces of the silica-based substrate utilizing an air pump for one to three minutes.

It is preferred to use devices with narrow inlet and outlet channels for capture and storage of nucleic acid. Narrow channels help to prevent environmental contamination, including contamination with nucleases, which are ubiquitous in the environment.

The present invention has multiple applications in laboratory research, human and veterinary medicine, public health and sanitation, forensics, anthropological studies, environmental monitoring, and industry. Nucleic acid extracted and stored according to the invention is readily used in a variety of downstream processes, including amplification, hybridization, blotting, and combinations thereof. The methods of the invention can be employed within point-of-care diagnostic assays to identify disease pathogens, and can be utilized in genetic screening. These devices and methods can also be used within veterinary medicine for the diagnosis and treatment of animals, including livestock and companion animals such as dogs, cats, horses, cattle, sheep, goats, pigs, etc.

The invention is illustrated by the following, non-limiting examples.

### EXAMPLES

### Example 1

Human DNA (Sigma Chemical Co.) was dissolved in 10 mM Tris-Cl, 1 mM EDTA pH 8.0 (TE) by gentle shaking overnight. The concentration was determined by reading the absorbance at 260 nm. 0.5 mg of DNA was digested in a total volume of 1 mL with 50 units of restriction enzymes Alu I and Mse I overnight at 37° C. Using the absorbance at 260 nm to monitor the concentration, the fragmented DNA was diluted to a final concentration of 50 μg/mL. The final concentration was confirmed by analysis with a commercially available fluorescent dye (PICOGREEN; Invitrogen Corporation, Carlsbad, Calif.) using a previously diluted batch of human DNA as a standard. The fragmented DNA was stored in aliquots at −20° C. Fragmented DNA appeared as a smear in agarose gel electrophoresis with a range of 50 to 400 bp.

The fragmented DNA was introduced into DNA cards at 500 ng per card. Each sample contained 0.5 mL TE, 10 μl of fragmented DNA, 0.5 mL GH Lysis Buffer (6M Guanidine hydrochloride, 50 mM citric acid pH 6.0, 20 mM EDTA, 3% Triton X-100, 10% Tween-20), and 0.5 mL ethanol. The entire sample was loaded onto a card which was then allowed to sit for at least 30 minutes at room temperature to allow adsorption of the DNA onto the glass substrate. The card was washed twice at room temperature with 1 mL each of Wash 1 (2M Guanidine hydrochloride, 33 mM sodium citrate pH 6.0, 6.7 mM EDTA, 33% ethanol). The cards were then washed six times with lmL each of Wash 2 (20 mM Tris-Cl pH7.0, 0.03% PROCLIN 150, 70% ethanol). Bound nucleic acid was eluted from the cards using 100-μl aliquots of TE.

Nucleic acid was quantitated by quatitative PCR (qPCR) using primers and probe specific for the CCR5 gene as shown in Table 1.

TABLE 1

| | |
|---|---|
| CCR5F | TACCTGCTCAACCTGGCCAT [SEQ ID NO: 1] |
| CCR5R | TTCCAAAGTCCCACTGGGC [SEQ ID NO: 2] |
| CCR5 | (6-FAM)TTTCCTTCTTACTGTCCCCTTCTGGGCTC(TAMRA) |
| Probe | [SEQ ID NO: 3] |

FAM is fluorescein and TAMRA is the fluorescence quencher.

The qPCR reaction mixtures contained 12.5 μl PCR Buffer (Roche), 0.075 μl of each CCR5F [SEQ ID NO:1] and CCR5R [SEQ ID NO:2], 0.05 μl CCR5 probe [SEQ ID NO:3], water to 20 μl, and 5μl of either human DNA standards or the card eluate. Reactions were run in an Applied Biosystems 7500Fast machine with a program consisting of 2 minutes at 50° C., followed by 45 cycles of 95° C. for 15 seconds and 1 minute at 60° C.

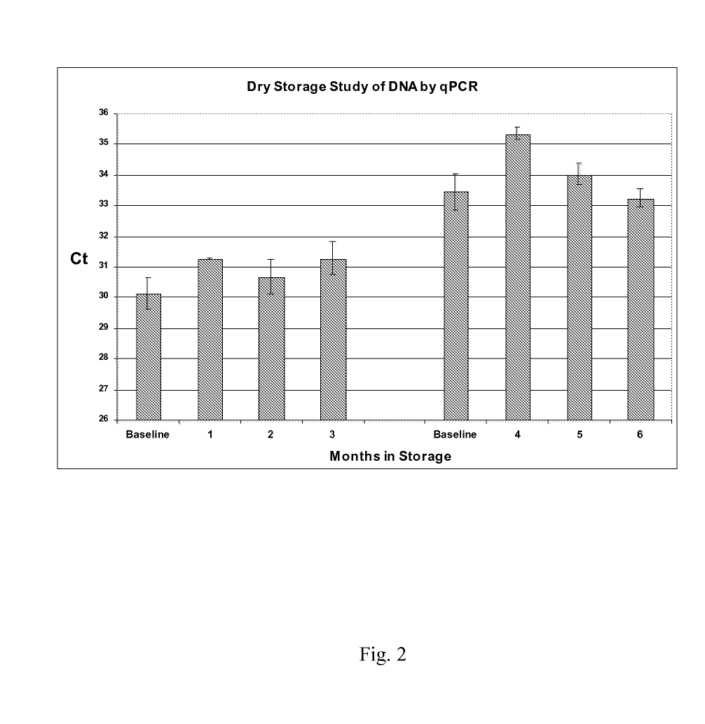

Results (as Ct) are shown in FIG. 2. Baseline indicates cards eluted at time zero. All other cards were stored at room temperature and eluted over a six-month period of time. Time points through 3 months were tested together and time points 4-6 months with baseline repeated were done at a separate time. Baseline was retested to account for variability in the PCR assay. The results show when compared to the baseline or baseline repeat that the bound DNA was recovered in an amount and quality equivalent to baseline over the entire study period. We conclude from this study that DNA can be stored and maintained dry and at room temperature over a minimum of 6 months on a DNA extraction card comprising an unmodified glass substrate.

### Example 2

Accelerated aging studies of stored DNA are carried out at elevated temperatures. Extrapolation to room temperature storage is based on the activation energy, which is related to the rate at which the DNA may degrade over time. Two published values for activation energy (Anderson G and Scott M (1991). Determination of Product Shelf Life and Activation Energy for Five Drugs of Abuse. Clin. Chem. 37: 398-402.) are used in modeling, although previous experience indicates that degradation of DNA is very slow at room temperature and the value of 10 for the activation energy is likely the correct one. Table 2 shows how many days of storage at elevated temperature are equivalent to one year at room temperature for each activation energy estimate.

TABLE 2

| Activation Energy (kcal mol⁻¹ K⁻¹) | | |
|---|---|---|
| | 10 | 20 |

| | | | | |
|---|---|---|---|---|
| 20° C. | 365 | days | 365 | days |
| 37° C. | 142 | days | 55 | days |
| 45° C. | 94.5 | days | 24.5 | days |
| 60° C. | 46 | days | 5.9 | days |

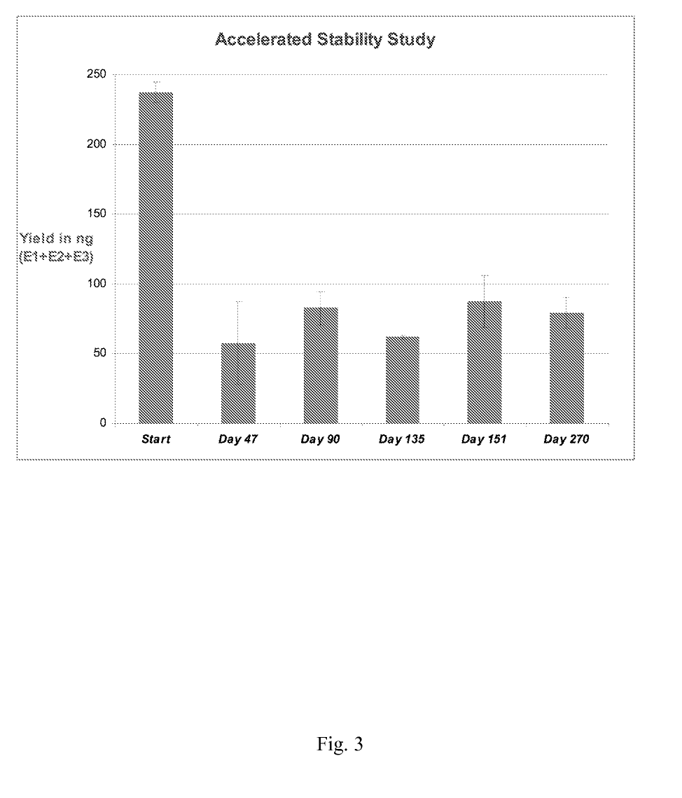

Samples (1.5 mL total volume) were prepared with one third volume each of water, lysis buffer (6M Guanidine hydrochloride, 50 mM citric acid pH 6.0, 20 mM EDTA, 10% Tween-20, and 3% Triton X-100), and ethanol. Each sample contained 500 ng of fragmented human DNA. DNA cards were loaded with the sample and allowed to sit for 1 hour to allow DNA adsorption to glass. Cards were washed twice with 1 mL Wash 1 (Example 1), and six times with Wash 2 (Example 1). Residual alcohol was dried from the surface of the glass under a stream of air. Bound nucleic acid was eluted in 200 μl TE (10 mM Tris-Cl, 1 mM EDTA pH 8.0). Cards were stored at 45° C. for the times indicated in FIG. 3. Eluates (three per card) were assessed for recovery of bound DNA by fluorescent analysis (using PICOGREEN dye, obtained from Life Technologies) and by qPCR using samples diluted 1:10 in water and the CCR5 gene primer set as disclosed in Example 1 [SEQ ID NO:1, NO:2, and NO:3]. All eluates were kept at −20° C. to allow reassay when necessary.

Data are shown in FIG. 3. Between the start and day 47, a large drop in recoverable DNA was observed. After that, recoverable DNA leveled off. While not wishing to be bound by theory, it is believed that the elevated temperature caused some of the DNA to become irreversibly bound the glass, while the remaining third could be recovered. Irreversible binding of DNA to glass is known and may be much greater at 45° C. than at room temperature. In this study, 94 days at elevated temperature corresponds to 1 year of storage at room temperature, so 270 days represents 3 years of storage.

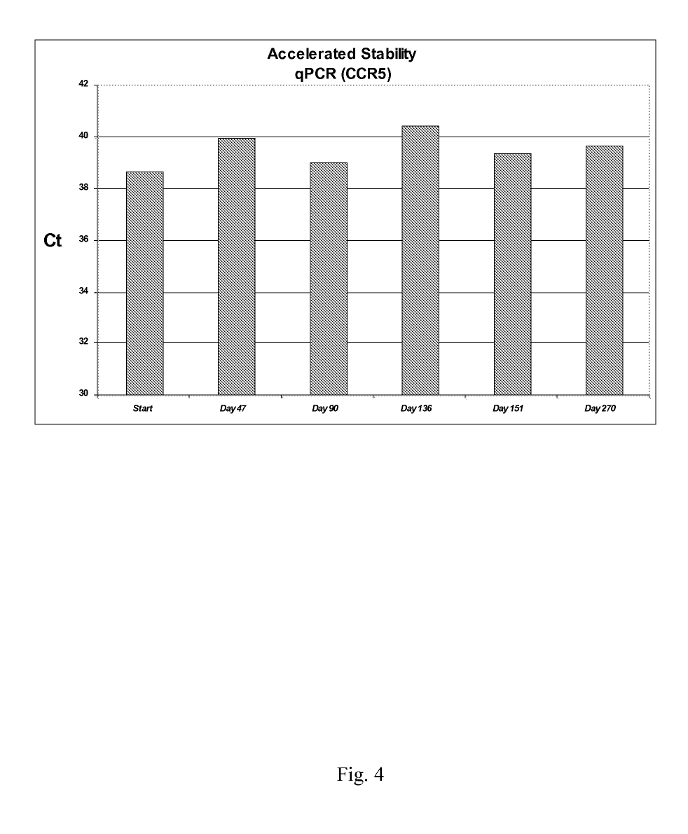

Results of the qPCR analysis are shown in FIG. 4.

### Example 3

The sample for testing stability of RNA bound to glass was bacteriophage MS2 RNA suspended in buffer supplemented with a non-functional carrier RNA. The non-functional carrier RNA was used to protect the much more dilute MS2 RNA from losses due to non-specific adsorption in handling and preparing the RNA for binding and to make the sample more uniform. It has been shown that carrier RNA may aid in recovery of greater quantities of target RNA (such as MS2) and does not have a negative effect on extraction.

The carrier, poly(rA), was added directly to the RNA diluent. A 200-μl aliquot of poly(rA) (Sigma-Aldrich, Inc.; dissolved at 5 mg/mL) was added to 5 mL of diluent (TE: 10 mM Tris-Cl pH 8.0, 1 mM EDTA).

MS2 RNA (obtained from Roche) was supplied at 800 ng/μl (4.1E12 copies/100). Serial dilutions to 4.1E7 copies/100 were prepared. 10 μl was used per binding mix.

The binding buffer contained 6M Guanidine hydrochloride, 50 mM citric acid pH 6.0, 20 mM EDTA, 10% Tween-20, 3% Triton X-100. Carrier RNA was added to the binding buffer (100 μl of 5 mg/mL RNA per 10 mL binding buffer). Samples contained 10 μl diluted MS2 RNA, 0.5 mL TE without carrier, 0.5 mL Binding Buffer with carrier, 0.5 mL ethanol. Samples (1.5 mL) were loaded onto cards. Cards were left at room temperature for 30 minutes to allow for adsorption of RNA. The cards were washed twice with 1 mL wash 1 (2M Guanidine hydrochloride, 33 mM sodium citrate pH 6.0, 6.7 mM EDTA, 33% ethanol), and six times with wash 2 (20 mM Tris-Cl pH 7.0, 0.03% PROCLIN, 70% ethanol). Excess ethanol was removed by drying the cards under a stream of air. Bound RNA was eluted with 100 μl TE. Functional stability was assessed by qRT-PCR.

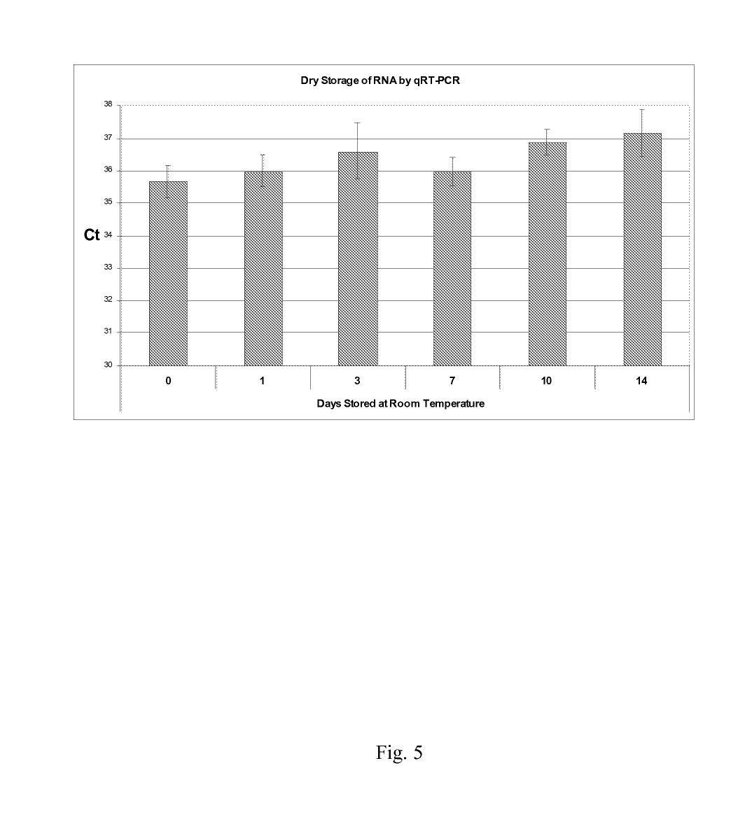

qRT-PCR was run in 25-0 total volume containing 12.5 0 reaction mix (Thermo-Fisher), 0.250 reverse transcriptase (MULTISCRIBE; supplied with Thermo-Fisher kit), 10 (10 μM stock) each forward and reverse primers (forward: CCTCAGCAATCGCAGCAAA [SEQ ID NO:4], reverse: GGAAGATCAATACATAAAGAGTTGAACTTC [SEQ ID NO:5]), 0.05 μl probe (100 uM stock, (6-FAM)CAAACATGAGGATTACCCATGTCGAAGACA(TAMRA) [SEQ ID NO:6]), water to 20 l. Each reaction mixture also contained 5 μl of card eluate or standard MS2 RNA. Samples were run using a program consisting of 30 minutes at 50° C.; 15 minutes at 95° C.; and 45 cycles, each consisting of 15 seconds at 95° C. and 1 minute at 60° C. Quantitation was accomplished relative to a series of serial ten-fold dilutions of purified MS2 RNA (Roche). The passive dye ROX, used by the thermocycler to normalize intra-well fluorescence, was added to all reactions at a concentration of 0.1 μM. Results, shown in FIG. 5, indicate that RNA bound to the glass substrate was highly stable for at least seven days.

### Example 4

A study was carried out to investigate the stability of DNA fragments bound to the walls of glass tubes and to the silica gel membrane capture substrate in spin columns.

Commercially available DNA was fragmented by restriction enzyme digestion. A large batch of sample was prepared in Guanidine hydrochloride buffer containing 250 ng DNA fragments per 0.75 mL sample. Extraction of DNA on glass was carried out essentially as disclosed in Example 1 using 4-mm (O.D.) glass tubing cut to 18-cm lengths, a length that holds 0.75 mL. Each tube was loaded with 0.75 mL sample, and the tubes were allowed to sit for 30 minutes, then washed twice with 0.5 mL Wash 1 (Example 1), and six times with 0.5 mL Wash 2 (Example 1). Tube ends were blotted on paper toweling, and the tubes were dried at 37° C.

Spin columns were obtained from Qiagen. 0.75 mL of sample was loaded on each column. The columns were spun for 1 minute at 8K RPM in a microcentrifuge. The columns were washed with 0.5 mL Wash 1 and 0.5 mL Wash 2 by pipeting the wash to the top of the column, and spinning in a microcentrifuge for 1 minute each. The columns were dried by spinning them at 14K RPM for 1 minute.

All samples were run in triplicate. Columns and tubes were stored in a box in a drawer at room temperature. DNA was eluted as in Example 1 using 3 elutions of 200 μl each. DNA was quantitated by analysis of eluates using fluorescent dye (PICOGREEN; Invitrogen Corporation, Carlsbad, Calif.). All samples were analyzed on the same day to assure that the results for each time point were directly comparable. Elution data (as DNA yield in ng) are shown in Table 3, wherein E1, E2, and E3 are the first, second, and third elutions, respectively; Avg is the average of three replicates; Stdev is the standard deviation; and Sum is the sum of the three elutions.

TABLE 3

| | | E1 | | E2 | | E3 | | |
|---|---|---|---|---|---|---|---|---|
| | Day | Avg | Stdev | Avg | Stdev | Avg | Stdev | Sum |
| Tube | 0 | 70.8 | 9.8 | 29.4 | 11.5 | 3.5 | 0.4 | 103.7 |
| | 91 | 42.5 | 7.4 | 21.3 | 5.5 | 5.1 | 0.9 | 68.9 |
| | 181 | 56.1 | 2.8 | 15.6 | 6.7 | 6.9 | 0.8 | 78.6 |
| | 272 | 40.9 | 5.0 | 9.1 | 3.0 | 2.5 | 1.3 | 52.4 |
| | 370 | 40.1 | 3.3 | 7.0 | 2.1 | 0.8 | | 47.9 |
| Column | 0 | 205.2 | 19.5 | 11.7 | 1.2 | 1.5 | 0.6 | 218.4 |
| | 91 | 178.6 | 2.4 | 8.3 | 1.2 | 3.8 | 1.6 | 190.7 |
| | 181 | 191.0 | 24.8 | 5.5 | 0.3 | 2.1 | 0.0 | 198.6 |
| | 272 | 163.5 | 17.1 | 6.1 | 0.7 | 2.0 | 1.0 | 171.6 |
| | 370 | 160.3 | 13.5 | 3.6 | 0.8 | 0.7 | 0.6 | 164.5 |

The trend for both tubes and columns was for lower recovery after 9 months of storage. For tubes, day 272 recovery was about half of the starting amount. For columns, the decrease amounted to about 20% of Day 0 at Day 272. The observed reduction may be due to degradation or to tighter binding to the glass over time as DNA dries further and becomes very slow to hydrate and disperse.

### Example 5

Bacteriophage MS2 was diluted in the amount of 1E4 phage per 0.5 mL of a commercially available universal viral transport medium (obtained from Becton Dickinson, Franklin Lakes, N.J.). 0.5 mL guanidine hydrochloride lysis buffer (Example 3) and 0.5 mL EtOH were added and mixed well. The sample was loaded onto DNA cards and allowed to sit for 20 minutes. Cards were washed and dried essentially as disclosed in Example 3, and stored as indicated. One set of triplicate cards for each time point was kept at room temperature while a parallel set was kept at −20° C. At each point, bound RNA was eluted from each card in 100 μl TE buffer. Card eluates were stored at −20° C. until assay.

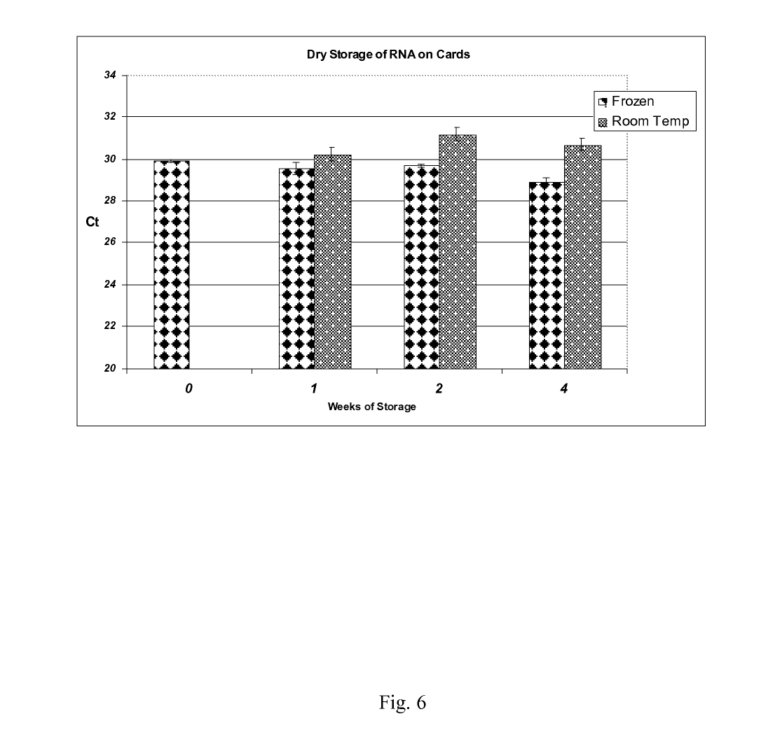

qRT-PCR was carried out using a commercially available kit (VERSO 1-Step QRT-PCR kit; Thermo Fisher Scientific, Rockford, Ill.) as directed by the manufacturer using 50 of each eluate for each PCR reaction and the primers and probe disclosed in Example 3 (SEQ ID NOS:4, 5, and 6). FIG. 6 shows a comparison of the recovery of MS2 RNAs from cards stored frozen and at room temperature over the course of 1 month. RNA kept frozen should be relatively stable from loss of function. As shown in the figure, functional RNA was recovered from cards stored frozen and at room temperature for up to one month. At one week, both storage conditions yielded RNA of equivalent functionality. At four weeks, RNA stored at room temperature lost some function as evidenced by a drop of approximately 1 Ct in the PCR profiles. This is a relatively small decrease given the ease of storage of dried bound RNAs.

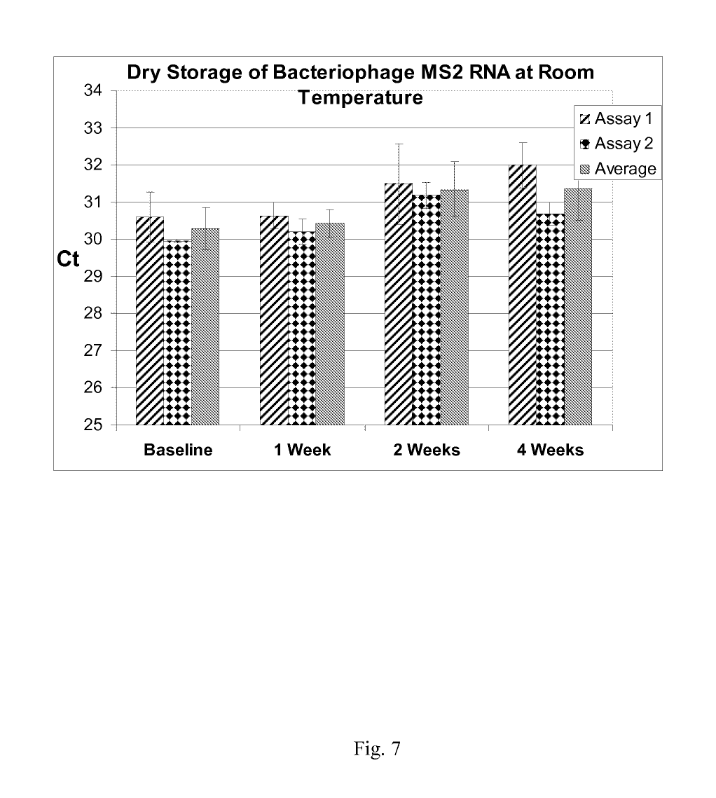

As a test of the reproducibility of the PCR assay, dry storage stability room temperature samples were re-assayed in the same PCR assay and compared to the previous day's assay. Those results are shown in FIG. 7. Day-to-day differences in PCR yield varied by roughly 0.5 Ct units. This result suggests that the functional differences between baseline and 4-week stability time points are relatively small and that functional RNA can be stored on flat glass cards for 1 to 4 weeks without significant loss of PCR signal.

What is claimed is:

1. A process for storing nucleic acid in a stable form, comprising:
applying a solution comprising nucleic acid to an unmodified, silica-based substrate whereby at least a portion of the nucleic acid binds to a surface of the substrate to produce bound nucleic acid;
washing the bound nucleic acid with a wash solution to produce substrate-bound, washed nucleic acid;
drying the substrate-bound, washed nucleic acid to produce dried nucleic acid; and
storing the dried nucleic acid on the substrate at a temperature of from 5° C. to 60° C. for a period of at least one week.

2. The process of claim 1 wherein the nucleic acid is DNA.

3. The process of claim 1 wherein the nucleic acid is RNA.

4. The process of claim 1 wherein the solution further comprises a chaotropic salt.

5. The process of claim 1 wherein the wash solution comprises an aliphatic C1 to C4 alcohol.

6. The process of claim 5 wherein the alcohol is ethanol.

7. The process of claim 5 wherein the alcohol is methanol.

8. The process of claim 1 wherein the washing step comprises washing the bound nucleic acid with a first wash solution and a final wash solution, wherein the final wash solution does not comprise a mineral salt.

9. The process of claim 1 wherein the silica substrate is glass.

10. The process of claim 9 wherein the glass is smooth glass.

11. The process of claim 10 wherein the smooth glass is a portion of an inner surface of a device, the device comprising the inner surface, an outer surface, a first port, and a second port, wherein the inner surface defines a binding chamber providing fluid communication between the first port and the second port.

12. The process of claim 1 wherein the substrate is a silica gel membrane.

13. The process of claim 1 wherein, following the storing step, the nucleic acid is eluted from the substrate.

14. The process of claim 13 wherein the eluted nucleic acid is amplified.

15. The process of claim 1 wherein the nucleic acid is stored for at least two weeks.

16. The process of claim 1 wherein the nucleic acid is stored for up to six months.

17. The process of claim 1 wherein the nucleic acid is stored for up to one year.

18. The process of claim 1 wherein the nucleic acid is stored for up to two years.

19. The process of claim 1 wherein the nucleic acid is stored for up to five years.

20. The process of claim 1 wherein the nucleic acid is stored at a temperature of from 15° C. to 30° C.

21. A process for storing nucleic acid in a stable form, consisting of:
applying a solution comprising nucleic acid to an unmodified, silica-based substrate whereby at least a portion of the nucleic acid binds to a surface of the substrate to produce bound nucleic acid;
washing the bound nucleic acid with a wash solution to produce substrate-bound, washed nucleic acid;
drying the substrate-bound, washed nucleic acid to produce dried nucleic acid; and
storing the dried nucleic acid on the substrate at a temperature of from 5° C. to 60° C. for a period of at least one week.


SEQUENCE LISTING (6 sequences)

SEQ ID NO: 1 - DNA, 20 bp, Artificial Sequence
  Synthetic
tacctgctcaacctggccat

SEQ ID NO: 2 - DNA, 19 bp, Artificial Sequence
  Synthetic
ttccaaagtcccactgggc

SEQ ID NO: 3 - DNA, 29 bp, Artificial Sequence
  Synthetic
nttccttcttactgtccccttctgggctn

SEQ ID NO: 4 - DNA, 19 bp, Artificial Sequence
  Synthetic
cctcagcaatcgcagcaaa

SEQ ID NO: 5 - DNA, 30 bp, Artificial Sequence
  Synthetic
ggaagatcaatacataaagagttgaacttc

SEQ ID NO: 6 - DNA, 30 bp, Artificial Sequence
  Synthetic
naaacatgaggattacccatgtcgaagacn